(19) United States
(12) Patent Application Publication
(10) Pub. No.: US 2012/0203241 A1
(43) Pub. Date: Aug. 9, 2012

(54) SINGLE PORT SURGICAL TISSUE CONTAINMENT AND EXTRACTION DEVICE

(75) Inventor: Warren P. Williamson, IV, Loveland, OH (US)

(73) Assignee: WW7 L.L.C., Marco Island, FL (US)

(21) Appl. No.: 13/501,540

(22) PCT Filed: Oct. 19, 2010
(86) PCT No.: PCT/US10/53143

§ 371 Date: Apr. 12, 2012

Related U.S. Application Data
(60) Provisional application No. 61/252,861, filed on Oct. 19, 2009.

(51) Int. Cl. A61B 17/28

(52) U.S. Cl. 606/114

(57) ABSTRACT

A tissue containment and extraction device including a tissue grasper configured to extend into a patient through an access site. The device includes jaws operative to grasp onto tissue to be extracted from the patient, and a tissue container having a distal end and a proximal end. The distal end of the grasper extends through the proximal end of the tissue container and is further operative to be extended through the distal end of the tissue container to grasp onto and pull the tissue proximally into the tissue container. A deployment structure is operatively coupled with the tissue container to allow a user to open the distal end of the container for receipt of the tissue and close the distal end of the container after the tissue is pulled into the tissue container. Related methods are also disclosed.

### CROSS-REFERENCE TO RELATED APPLICATIONS

This application claims the benefit of U.S. Provisional Application Ser. No. 61/252,861 filed on Oct. 19, 2009 (pending), the disclosure of which is hereby incorporated by reference in its entirety.

This generally relates to U.S. Pat. Nos. 6,610,072 and 6,152,936, the disclosures of which are fully incorporated by reference herein.

### BACKGROUND

This Invention relates generally to the art of surgery and in particular to the art of removing tissue from the patient during a surgical procedure. Many modern surgical procedures utilize small incisions or access sites through which endoscopic instruments are inserted to perform surgery in a minimally invasive manner. In some procedures it is necessary to ligate and remove tissue from a patient.

Quite often during a surgical procedure, tissue specimens are severed within the body cavity and then must be removed from the patient for diagnostic examination or for other reasons. For proper diagnosis it is imperative that the tissue in question be removed intact with as few lacerations or cuts as possible. This is because the pathologist must be able to report healthy tissue margins around diseased tissue. It is extremely difficult to try to reassemble any tissue which has been sectioned or mutilated prior to arrival at the histopathology lab. The surgeon removing the tissue is rarely in a position to help the pathologist try to re-assemble removed tissue bits. Therefore, it is advantageous that any tissue sample be removed from the body intact for diagnostic examination. However, this presents a particular problem when the incisions used for body cavity access are extremely small.

In minimally invasive procedures the surgeon uses the smallest access incision possible. However, if a tumor or other disease structure is found which needs to be biopsied or removed in its entirety, these incisions can be enlarged to a size just big enough to allow the tissue to be squeezed through the opening and removed from the patient cavity. This can create some cause for concern. For instance, if the tissue sample were to be cancerous in nature there have been studies that have shown that cancerous cells can be transferred to the access site while straining to pull the tumor through a small hole in the tissue. In such situations it is now common practice to attempt to enclose the tumor in a polyethylene bag while the excised tissue is still within the body cavity. Some advocate installing a plastic tube into the incision and drawing the tissue through the tube to protect the wall. When removing the tumor, the bag or tube becomes a barrier, which does not permit cells to dislodge and seed the patient's access site with cancer cells. As surgical instruments become smaller and smaller in diameter, it becomes more of a challenge for them to manipulate larger tissue samples.

While there are existing prior art “bag on a stick” type devices, they are very cumbersome to use. These devices, known as Endo-bags, are used to capture tissue and remove it from the patient. An Endo-bag is nothing more than a plastic bag with a purse string closure where the purse string tail is encased in a rigid plastic tube to facilitate deployment inside a body cavity. It is also important to understand how difficult it is to push a sample into a flaccid bag and close the bag without snagging the closure device on the grasper. In today's surgical procedures it is more and more commonplace to have only one surgical incision site. With more than one incision site the Endo-bag is somewhat easier to use because a separate grasper can be positioned desirably relative to the bag. That makes manipulation of tissue and insertion into the bag somewhat easier. However if the bag and grasper need to be in the same incision port—in other words parallel and in close proximity—it is almost impossible to manipulate the tissue into the bag and then close the bag. In addition it is very difficult to remove the bag without destroying the integrity of the bag while still inside the patient. The Endo-bag is also very difficult to direct through a trocar without tearing the bag. It would therefore be advantageous to provide a grasper and bag continuation system which makes a single port tissue extraction process easy and fast with less chance of contamination to the patient during extraction of the tissue sample.

FIG. 21 shows a prior art configuration known as an Endo-bag which utilizes a flexible bag with a purse string suture loop 107 around the opening 106 of the bag 101 terminating in a slip knot 108. The suture tail 109 is passed through a hollow tube 110 and extends out the proximal end of the tube 110. The bag 101 is folded around the hollow tube 110 and inserted into the body cavity through a trocar (not shown). Graspers (not shown) are used to open the purse string suture 107 to gain access to the inside of the bag 101 once inside the body cavity. However, any pulling action on the suture 109 results in the opening 106 of the bag 101 closing in on itself into a flattened oval rather than opening it out into a full circle. This makes insertion of the tissue sample into the bag 101 difficult. Release of the tissue sample into the bag 101 and removal of the grasper from the bag 101 are also difficult.

### SUMMARY

The preferred embodiment of the disclosed device has a unique system for drawing the tissue into a tissue container, such as a tissue sample bag, by use of a grasper, such as a mechanical grasping structure or a suction wand. The grasper extends through the proximal end of the bag and an opening deployment structure is provided for opening and managing the distal end of the bag. The opening deployment structure assures a wide distal opening to obtain a large tissue access opening for the given bag size. In addition the deployment structure may act as a squeezing structure such as fingers to express out any unwanted fluids making the tissue as small as possible without harming the diagnostic integrity of the tissue during the extraction process. The device may be used in many ways such as, for example, extracting tissue samples for histopathological analysis or diagnosis, or for purposes of extracting tissue during other surgical procedures.

The device comprises a tissue container with proximal and distal ends and a wall, which may be generally cylindrical, between the proximal and distal ends. The proximal end has an elastic or otherwise grippable orifice. The grippable orifice may be a silicone O-ring or could be a small engagement element such as a purse string. This grippable orifice is adapted to be releasably retained on the shaft of the grasping tool used to introduce the tissue into the container, such as a flexible bag.

In its final assembly configuration, a mechanical grasper or a suction wand is inside the bag at the beginning of the procedure where it is positioned to be able to evert the bag (that is, turn the bag inside out) and draw the tissue into the proximal end of the bag. At the end of the procedure the grasper is withdrawn through the grippable orifice of the bag, which then closes on its own, or is otherwise closed, effectively sealing the proximal end of the bag. In an alternative embodiment, instead of the elastic orifice, a purse string tail could be pulled to tighten and close the proximal end of the bag.

The distal end of the bag incorporates a filament purse string attachment or closure around the perimeter of the opening. Around the perimeter of the bag are access scallops which allow deployment structure such as legs to snap onto the purse string element. In order for the device to be inserted through the abdominal or thoracic cavity the tissue bag is nested inside a hollow cavity created by the closing of the deployment structure. This creates the smallest circular cross-section so that the device may be introduced through a trocar into the patient. Once inside the patient, the grasper is advanced forward or distally, opening the deployment structure which, in turn, unfurl the tissue bag by pulling radially outward on the peripheral purse string filament.

Once a tissue bag is deployed and open, in one embodiment, four deployment structure legs hold the bag open to allow for easy access to the interior of the bag. The distal ends of the grasper jaws are now exposed at the proximal end of the tissue bag. As the grasper is further advanced through the proximal elastic or grippable orifice, the bag becomes everted. By further extending the grasper distally beyond the bag opening, one assures that by grasping the tissue and pulling it backwards or proximally into the bag opening that the tissue will remain at the deepest proximal point in the bag to allow full engagement inside the bag. The tissue may then be severed from whatever structure to which it is attached. The next step is to enclose the tissue in the bag. The distal purse string tail extends through the shaft of the tissue capturing device such as a cannula type structure into free space outside the patient's body. The surgeon grasps the handle of the tissue capturing device and pulls on the purse string tail. This closes the purse string at the distal end of the tissue bag. The deployment legs are releasably retained on the purse string with C-shaped snap type fittings. The legs, which are flexible, will track with the purse string as the purse string reduces in diameter. This action of the legs squeezes the contents of the bag and expressing any fluids. In the preferred embodiment the legs compress the tissue and hold the bag tightly enough to extract the sample from the body while still being enclosed by the legs. The legs help to guide the sample through the body wall.

As one alternative, once the purse string closure has become small enough, the snap fittings on the deployment legs will let go and release the bag from the instrument. At that point the grasper can be retracted fully, removing it from the proximal end of the bag through the elastic orifice. The elastic retainer, such as an O-ring, will close the hole which was left by the grasper. Now the tissue is inside the bag and is free from the device. The device may be removed from the patient through the trocar body. At the end of the procedure, after the trocar is removed, the surgeon will pull on the tail of the purse string and remove the bag containing the tissue through the patient's access site bringing it outside the body cavity.

In another alternative, a suction wand, in place of a mechanical grasper is used to draw the tissue into the bag. Once the tissue is drawn into the bag, the tissue can be released and the suction wand can be re-extended through the purse string opening to gather other tissue samples that need to be collected. One way retention features on the deployment legs, that extend into the inside of the bag, may be used to retain the tissue samples collected first and do not allow them to follow the suction wand out of the bag again as it is extended to collect other samples.

Various embodiments of this invention provide a tissue enclosing apparatus which can easily grasp and hold the subject tissue sample while facilitating the transfer of the tissue into a container through a single incision access port. The container can then be sealed off such as through use of a purse string type closure. With the grasper removed from the central lumen, the device will collapse onto the container facilitating withdrawal through the body cavity wall. Alternately, the container and tissue (contained inside) can be released from the device using the tail of the purse string that extends through the access site.

### DESCRIPTION OF THE FIGURES

FIG. 1 shows the tissue specimen removal device in a collapsed state, ready to be inserted into a body cavity.

FIG. 2 shows a grasper advancing forward and opening the supporting or deployment legs at the distal end of the device. The grasper resides inside the proximal end of the sample bag at this point.

FIGS. 3 through 9 depict only two of the four supporting legs to more clearly show the shape change of the bag during actuation.

FIG. 3 depicts a grasper as it is advanced beyond the deployment legs which everts the bag from the proximal end through the distal purse string closure. This action turns the bag inside out.

FIG. 4 shows the grasper taking hold of a tissue sample with the jaws extended distally beyond the confines of the sample bag.

FIG. 5 shows the grasper retracting through the tool core or lumen, with tissue sample attached, pulling the sample into the bag.

FIG. 6 shows the tissue sample fully retracted into the tissue sample bag surrounded by the deployment legs.

FIG. 7 shows the step of pulling on the distal purse string to flex the supporting legs and close the bag behind the tissue sample at the distal end. At this point the entire instrument including the tissue bag with its enclosed sample can be pulled out through the access site.

FIG. 8 shows an alternative embodiment whereby the supporting legs are snapped free by further pulling on the purse string tail. In addition the grasper is pulled proximally through the proximal port of the bag. This sets the bag free from the instrument.

FIG. 9 shows pushing the grasper forward or distally as one way to free the bag with its enclosed tissue sample from the instrument deployment legs.

FIG. 10 shows a perspective view of FIG. 2 and a set of equally spaced deployment legs around the periphery of the distal purse string. This figure shows the large distal opening of the tissue sample bag.

FIG. 11 is a perspective view equivalent to FIG. 4 showing the bag everted and the tissue grasper extended beyond the purse string and grasping a tissue sample.

FIG. 12 shows an equivalent perspective view of FIG. 5. The tissue is being drawn into the purse string opening of the tissue sample bag, while the bag is being withdrawn into the supporting legs by the grasper and tissue.

FIG. 13 is a perspective view equivalent to FIG. 6 whereby the supporting legs bend inward with the tightening purse string and express fluids and air from the tissue sample bag prior to removal.

FIG. 14 shows the preferred embodiment after the step depicted in FIG. 13. The bagged tissue sample remains captured by the supporting legs in preparation for removing the tissue sample from the patient.

FIG. 15 shows the tissue sample being drawn through the body cavity wall by extracting the instrument and the tissue sample simultaneously while the bag prevents contamination of the tissue wall.

FIG. 16 is a perspective view equivalent to FIG. 9 and shows a tissue sample being freed from the distal end of the device in preparation for removing the bag and tissue sample.

FIG. 17 shows an alternate embodiment whereby the tissue sample bag has been freed from the instrument and can be drawn through an opening in the patient's body cavity wall for extraction from the patient.

FIG. 18 shows the tissue sample bag with the purse strings at the larger distal end, and the self-closing orifice which accepts the grasper shaft at the proximal end of the bag.

FIG. 19 is a longitudinal cross sectional view of the bag depicted in FIG. 18.

FIG. 20 shows a bag such as one constructed in similar fashion to that in FIG. 18 however with the improvement of having woven filaments within the bag for extra strength.

FIG. 21 is a perspective view of a prior art endo-bag.

FIG. 22 shows a tissue capturing device in an alternate embodiment using a suction wand as the grasper and including one-way retention features molded onto the deployment legs near each of the C-shaped slots that hold the suture. The retention elements fan out during deployment of the support legs and provide a hindrance to material coming out of the bag once placed within it.

FIG. 23 shows an isolated perspective view of the deployment legs with the one-way retention features.

FIG. 24 is a perspective view of another embodiment showing a deployment leg with a differently configured one-way retention feature.

FIG. 25 shows the suction wand extended through the purse string suture opening of the bag and grasping a piece of tissue which will be drawn into the bag past the one-way retention features.

FIG. 26 shows the suction wand re-extended through the purse string suture opening of the bag in order to retrieve another piece of tissue. The first piece of tissue is captured within the bag and retained at the proximal end of the bag by the one-way retention features as the suction wand is moved on by.

### DETAILED DESCRIPTION OF THE ILLUSTRATIVE EMBODIMENTS

The instrument includes two basic components—the first being the tissue capturing and containing device; the second is a tissue grasper. Both have elongated shafts and handles at the proximal end of the instrument with tissue engaging features at the distal end. The tissue capturing and containing device has a hollow shaft with a central lumen which accepts the tissue grasper. The proximal end of the tissue grasper device has grip handles to facilitate manipulation of the distal end by use of one of the user's hands at the proximal end. Preferably, the grasper comes packaged with the device so that it interfaces well with the self-closing orifice at the proximal end of the tissue bag.

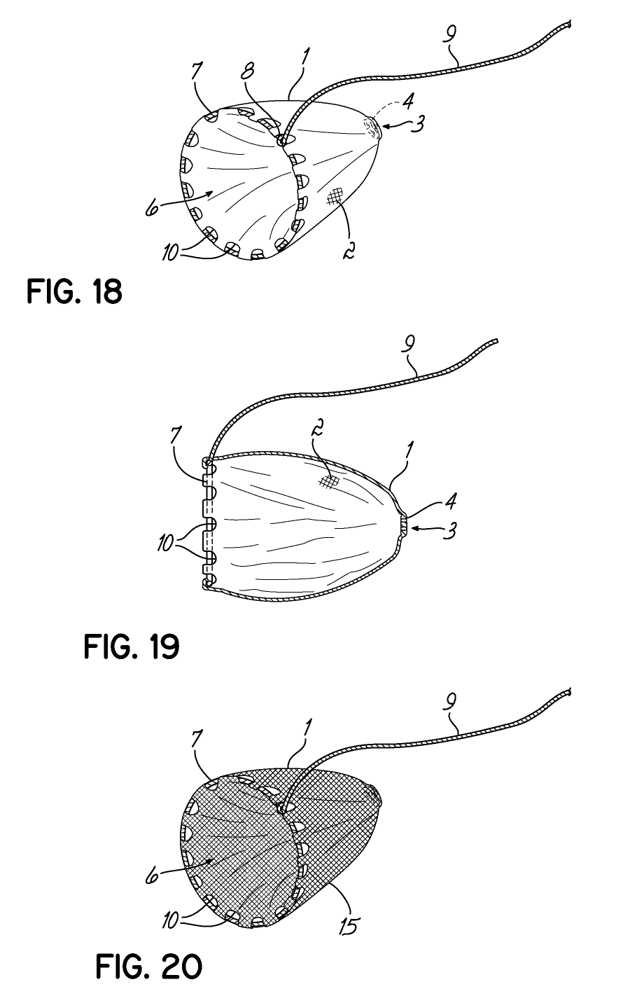

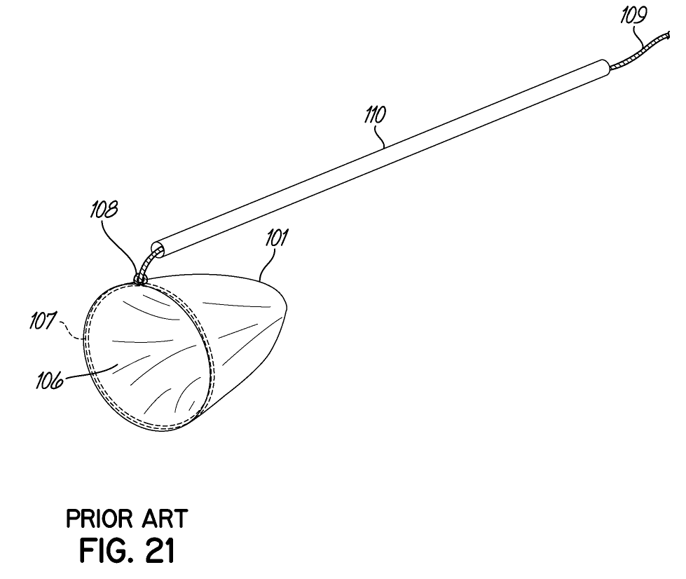

The tissue capturing bag 1 such as that shown in FIG. 18 consists of a thin barrier material 2 in the shape of a bag or pouch made from a material such as polyethylene, latex, urethane, synthetic rubber, or similar materials which provide both a barrier and enclosing structure. The bag has a proximal and distal end. The proximal end is closest to the user and incorporates an orifice 3. This orifice 3 is capable of being closed, either in a self-closing manner or by the user. The self-closing feature can be an O-ring or other elastic type closure 4. Alternatively, another purse string type closure is incorporated into the proximal end of the bag. The inside diameter of the orifice 3 is sized to releasably engage or grip the shaft of the grasper being deployed through the central lumen of the tissue grasping device. The distal end 6 of the bag 1 is a larger opening and incorporates a purse string type closure 7 using a single filament that is slip knotted 8 to provide a garrote (or purse string type closure structure 7). The tail 9 of the purse string extends proximally through the instrument body and outside of the patient's body cavity. Equally spaced around the distal opening 6 of the bag 1 are slotted openings 10 which facilitate snapping of the purse string element 7 onto the deployment legs 20 and also eliminate bunching of the bag material when the purse string 7 is closed. FIG. 19 is a cross section of FIG. 18. FIG. 20 shows an alternative embodiment of the bag described in FIG. 18, where bag 15 incorporates mesh elements either instead of or in addition to the bag material. There may be some instances where a mesh is a more desirable capturing barrier. It may also be advantageous to mix both a mesh structure and a film bag whereby the mesh helps to compress the tissue for extraction and the bag provides a barrier against cell seeding. This mesh material may be made out of woven monofilament such as nylon or polypropylene.

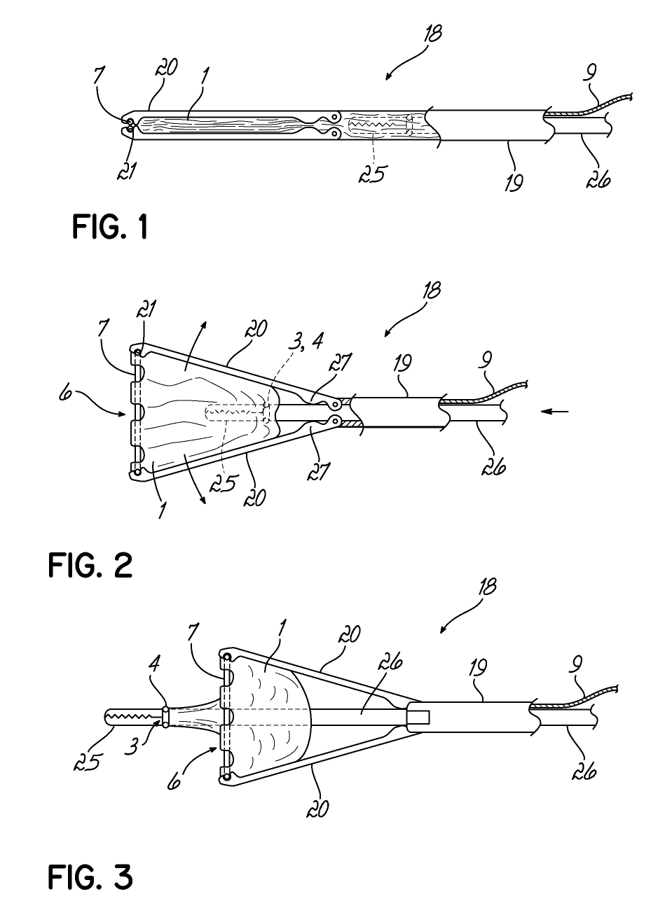

FIG. 1 shows a partial cutaway of the distal end of the tissue containment and extraction device 18 with the deployment legs 20 nested together and the tissue sample bag 1 retained within a central cavity created by the support or deployment legs 20 within a nested position. Suitable similar structures are shown and described in U.S. Pat. Nos. 6,152,936 and 6,610,072, incorporated by reference herein. Grasper 26 with jaws 25 are positioned behind the support legs 20 but still inside the proximal end of the tissue bag 1. The hollow shaft of the instrument 19 allows the tail 9 of the tissue bag purse string 7 and the shaft of the grasper 26 to be placed inside the central lumen of the instrument 19. The extended tail 9 is pulled for actuation and closure of the purse string 7 at the distal end of the bag 1.

In FIG. 2, as the grasper item 26 is advanced forward through the central lumen, the tip of the grasper 26 contacts cam points 27 on the supporting legs 20 pushing each supporting leg 20 away from the central axis of the shaft 19, and opening the distal purse string end of the bag 1 as the support legs 20 are pushed outward. A C-shaped slot 21 on supporting leg 20 allows the purse string filament 7 to be snapped into place and retained during the procedure. However, when the purse string 7 is close to its smallest size, it will be pulled free from the opening of the C-shaped slot 21, releasing the bag 1 from the supporting legs 20.

FIG. 3 shows a grasper 26 being further advanced through the distal end of the instrument and purse string 7 and distal opening 6 of the bag 1. The grasper jaws 25 are extended beyond the self-closing orifice 3. Notice that the self-closing orifice end 3 of the bag 1 has been everted through the opening of the purse string 7 and now extends axially outward from the instrument supporting legs 20, with the grasper jaws 25 exposed.

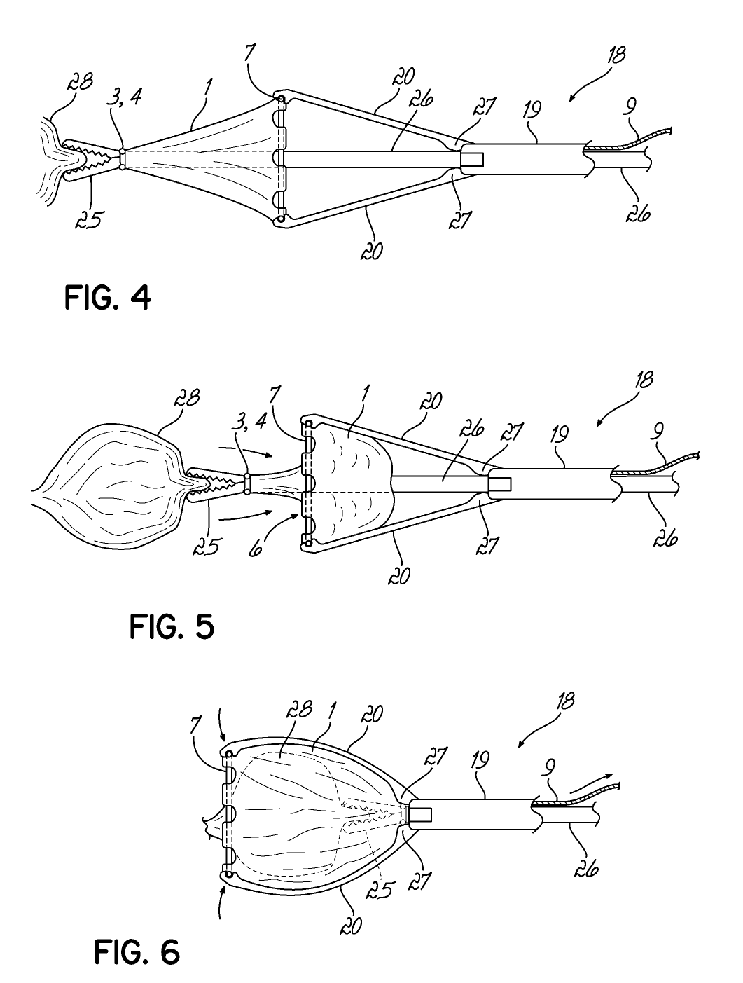

FIG. 4 shows a grasper 26 fully extended with jaws 25 engaging the tissue specimen 28. The bag 1 is fully everted and extended beyond the supporting legs 20.

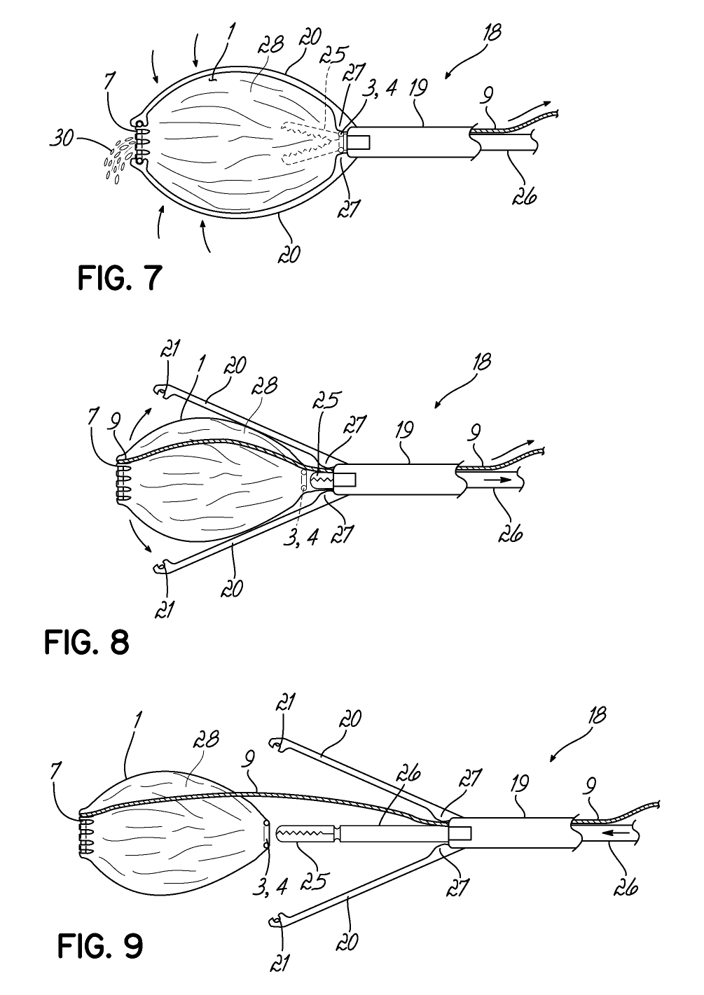

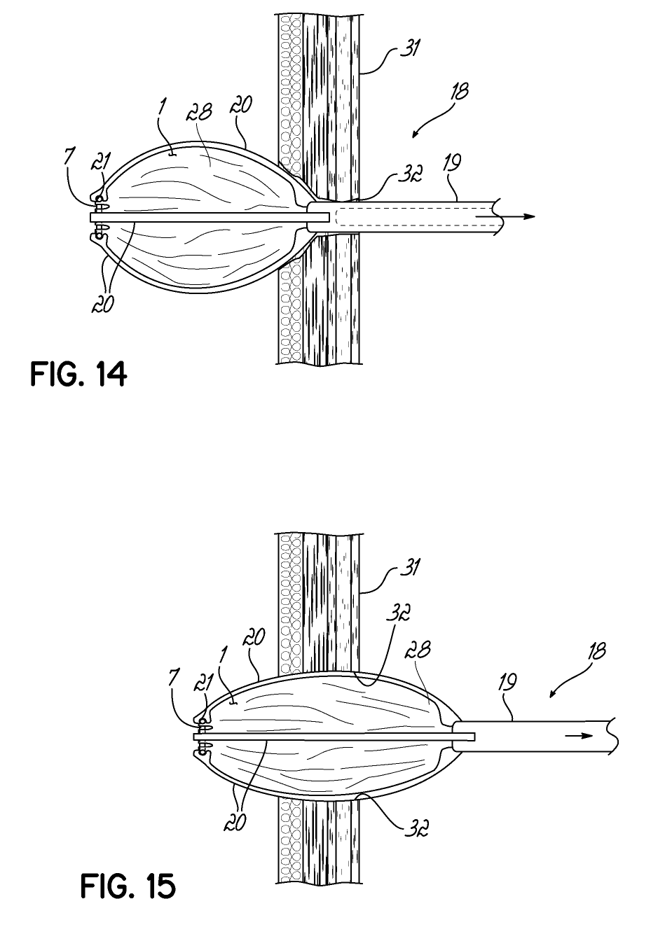

FIG. 5 shows the grasper 26 being retracted with the bag 1 and the tissue 28 re-entering the purse string 7. The supporting arm structure 20 and, more specifically, the supporting leg cams 27 continue to maintain the supporting legs 20 in the open position. Once a retractor has pulled the tissue 28 fully within the confines of the bag 1 such as in FIG. 6, a partial closure of the bag 1 by pulling on the tail 9 of the purse string 7 will flex support legs 20 radially inward at the distal end preventing the tissue 28 from falling out of the bag 1 during the next step. FIG. 7 shows the purse string tail 9 being pulled down slightly tighter to express any unwanted fluids 30 in the bag 1. Grasper jaws 25 are then relaxed from their grip on the tissue 28 inside the bag 1. At this point in a procedure, the user may decide to either pull the instrument and tissue through the body cavity wall together or to drop off the tissue bag 1 from the instrument and remove it at a later time. In the majority of cases where large tissue is captured in the bag 1, the procedure will proceed as shown in FIG. 14 where the tissue 28 encased in the bag 1 and the instrument are removed as one unit.

FIG. 14 shows the tissue supporting legs 20 surrounding the bag 1 and tissue sample 28. Supporting legs 20 further provide a guiding structure to prevent the tissue 28 from being dislodged from within supporting legs 20 during extraction from the body walls of the patient 31 through incision 32 made for access to the surgical site. Note that the purse string 7 still remains engaged in the releasable slot 21 of the supporting leg 20. The tail 9 of the purse string 7 will then be pulled fully tight to release the purse string 7 from the legs 20 once the instrument and tissue bag 1 are extracted from the patient.

FIG. 15 shows the distal end of the instrument with supporting legs 20 and tissue bag 1 passing through the surgical site 32.

If the user wishes to separate the bag 1 containing the tissue 28 from the instrument within the body cavity, the procedure would be as depicted in FIG. 8. Suture tail 9 is pulled hard enough to release C-shaped slots 21 at the end of each support leg 20 from purse string 7. The grasper 26 is then pushed distally as shown in FIG. 9 to release the tissue bag 1 and sample 28 distally from within supporting leg structure 20.

FIG. 17 shows an example of the alternate embodiment extracting the tissue bag 1 and sample 28 through the surgical incision 32 without the use of the distal end of the extraction tool.

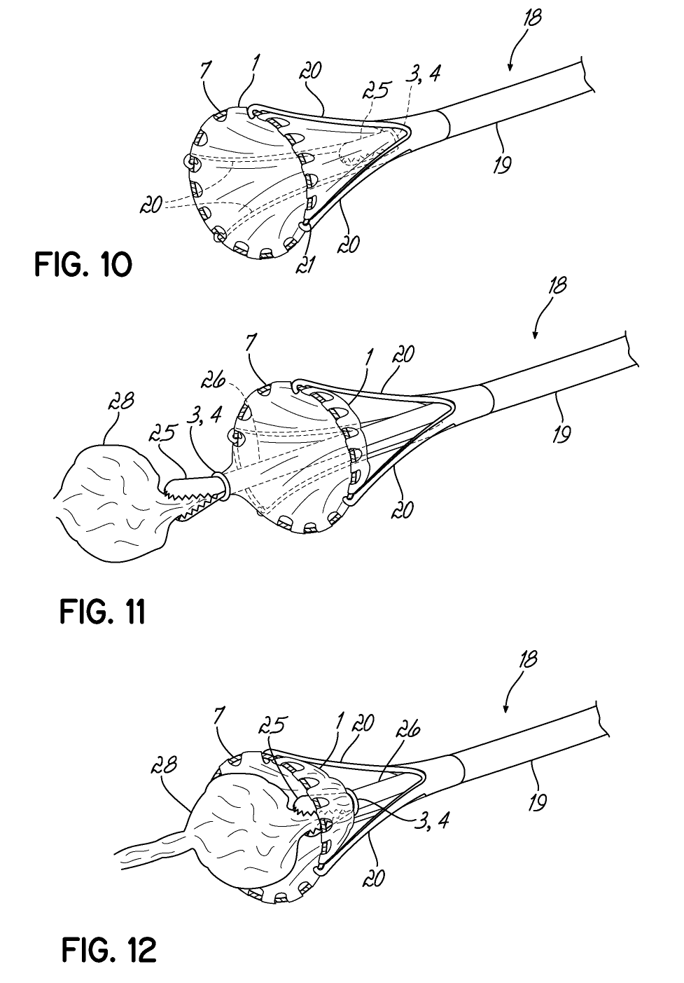

FIGS. 10, 11 and 12 show isometric views which give a better depiction of how the deployment legs 20 extend radially outward from a central axis of the device; each leg 20 supports a quadrant of the large opening at the distal end 6 of the bag 1.

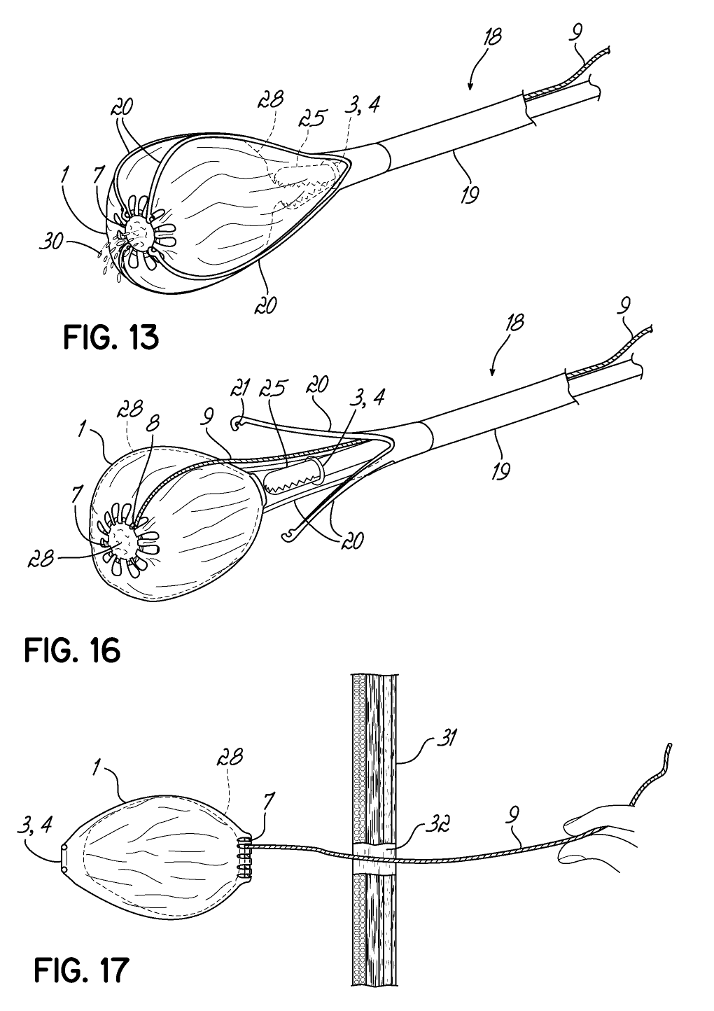

FIG. 13 is analogous to FIG. 7 showing full isometric view of the contents being expressed from the bag 1 during closure of the purse string 7. FIG. 16 is analogous to FIG. 9 which shows the tissue 28 and bag 1 being ejected from the support structure 20 at the distal end of the grasping instrument 26.

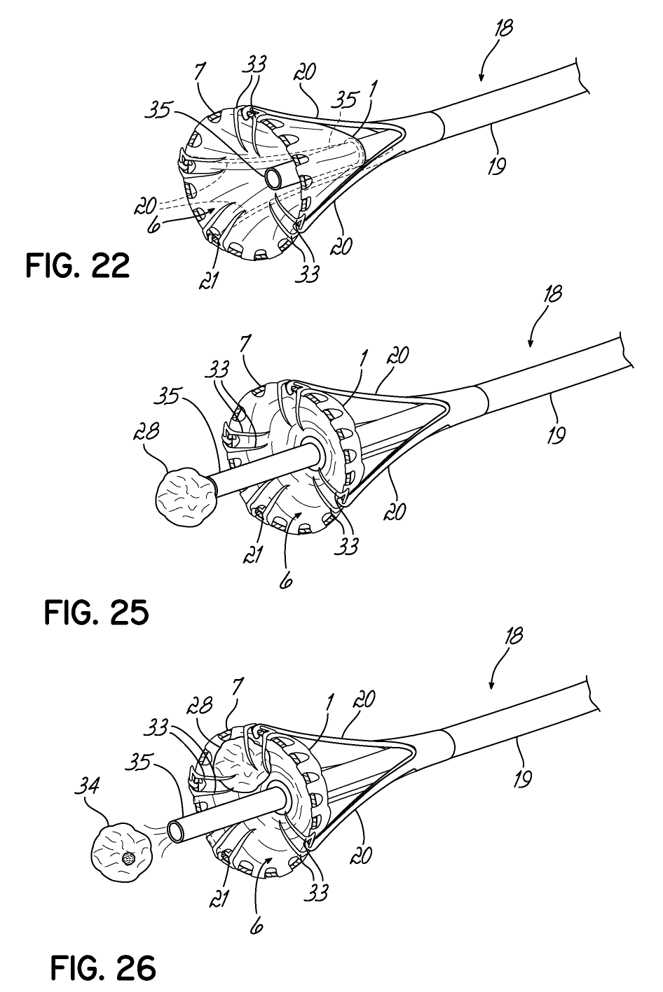

In FIG. 22, a suction wand 35 is used instead of a mechanical grasper as the tissue retrieval tool. This may be considered a vacuum operated grasper, and grasps tissue by applying suction to the tissue at the distal end of the wand 35. The suction wand 35 has advanced forward or distally through the central lumen of the device and this pushes each supporting leg 20 away from the central axis of the shaft 19. As the legs 20 expand, the distal purse string 7 opens end 6 of the bag 1. One-way retention elements 33 are molded to the supporting leg 20 near the C-shaped slot 21. They allow tissue 28 to be drawn in past them into the bag 1 but act to deter tissue 28 from exiting the bag 1 at the purse string suture 7 opening 6.

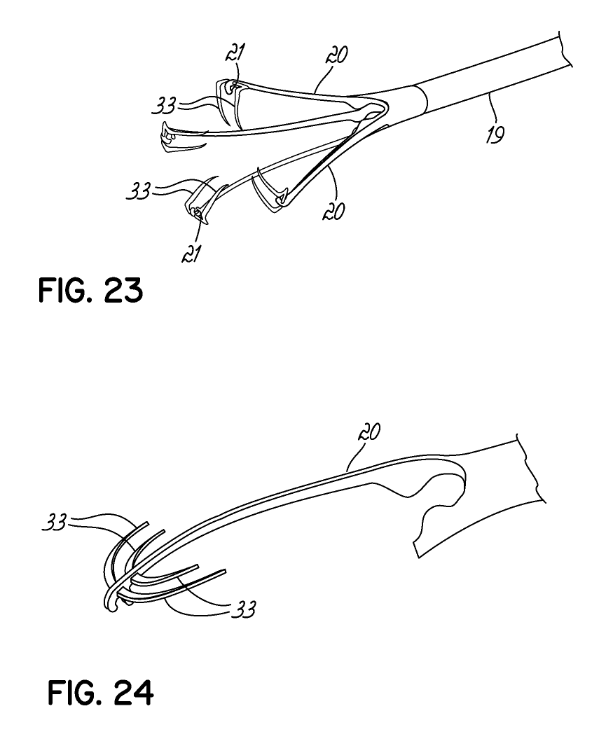

FIG. 23 shows the retrieval device without the collection bag but showing the four supporting legs 20 with one-way retention features 33 exposed. The one-way retention elements 33 are molded to the supporting leg 20 near the C-shaped slot 21 on each leg. These elements 33 are thin and flexible to allow them to fold inside the bag 1 when the supporting legs 20 are in the closed position prior to deployment. Elements 33 fan out when the deployment legs 20 are opened.

FIG. 24 shows a single support leg 20 with one-way retention features 33. In this example four one-way retention elements 33 are molded onto each supporting leg 20. Any number of one-way retention elements can be molded onto each leg in order to best retain collected tissue in the proximal end of the bag.

FIG. 25 shows a suction wand 35 further advanced through the distal end of the instrument and purse string 7 and distal opening of the bag 6. The distal end of the suction wand attaches to the tissue 28 ready to draw it into the bag past the one-way retention features 33.

FIG. 26 shows the first tissue sample 28 secured by the one way retention features 33 at the proximal end of the bag, while the suction wand 35 is re extended past the purse string suture 7 opening 6 of the bag to retrieve a second tissue sample 34.

While the present invention has been illustrated by a description of various preferred embodiments and while these embodiments have been described in some detail, it is not the intention of the Applicant to restrict or in any way limit the scope of the appended claims to such detail. Additional advantages and modifications will readily appear to those skilled in the art. The various features discussed herein may be used alone or in any combination depending on the needs and preferences of the user. This has been a description of illustrative aspects and embodiments the present invention, along with the preferred methods of practicing the present invention as currently known.

What is claimed is:

1. A tissue containment and extraction device, comprising:
a tissue grasper configured to extend into a patient through an access site, and including a distal end operative to grasp onto tissue to be extracted from the patient,
a tissue container having a distal end and a proximal end, the distal end of the grasper extending through the proximal end of the tissue container and further operative to be extended through the distal end of the tissue container to grasp onto and pull the tissue proximally into the tissue container, and
deployment structure operatively coupled with the tissue container to allow a user to open the distal end of the container for receipt of the tissue and close the distal end of the container after the tissue is pulled into the tissue container.

2. The device of claim 1, wherein the tissue grasper further comprises a mechanical grasping structure.

3. The device of claim 1, wherein the tissue grasper further comprises a suction wand.

4. The device of claim 1, wherein the tissue container further comprises a flexible bag providing a barrier to transmission of tissue cells during extraction of the tissue from the patient.

5. The device of claim 1, wherein the proximal end of the tissue container is configured to grip the tissue grasper.

6. The device of claim 5, wherein the proximal end of the tissue container is further configured to self-close upon removal of the tissue grasper from the proximal end of the tissue container.

7. The device of claim 1, wherein the tissue container turns inside out as the grasper is moved distally to grasp tissue and the tissue container returns to a normal configuration upon retraction of the grasper into the tissue container with tissue to be contained in the container and extracted from the patient.

8. The device of claim 1, wherein the deployment structure further comprises squeezing structure operative to reduce the size of the tissue and/or container prior to extraction from the patient.

9. The device of claim 1, wherein the distal end of the tissue container further comprises a purse string closure, wherein a tail of the purse string may be pulled by the user to close the distal end of the bag prior to extraction of the tissue and container from the patient.

10. The device of claim 9, wherein the purse string closure is releasably secured to the deployment structure to allow the tissue container and tissue contained therein to be removed as a separate unit from the patient.

11. The device of claim 1, further comprising a plurality of one way tissue retainers carried on the deployment structure and adapted to retain tissue within the container after the grasper has pulled the tissue proximally past the tissue retainers and into the tissue container.

12. A method for extracting and containing tissue from a patient, comprising:
directing a tissue grasper into a patient through an access site of the patient and through a tissue container positioned within the patient,
opening a distal end of the container,
extending a distal end of the grasper beyond the distal end of the container,
grasping tissue with a distal end of the grasper,
pulling the tissue into the tissue container with the grasper,
closing the distal end of the container with the tissue contained therein, and
extracting the tissue container and tissue contained therein from the access site.

13. The method of claim 12, wherein grasping the tissue further comprises using a mechanical grasping structure or a suction wand.

14. The method of claim 12, further comprising:
gripping the tissue grasper at a proximal end of the tissue container.

15. The method of claim 14, wherein extending the distal end of the grasper further comprises:
turning the container inside out as the grasper is extended distally.

16. The method of claim 14, further comprising:
removing the grasper from the proximal end of the container, and closing the proximal end of the container.

17. The method of claim 16, wherein closing the proximal end of the container further comprises:
self-closing the proximal end of the container.

18. The method of claim 12, further comprising:
squeezing the tissue and the container after the tissue is pulled into the container.

19. The method of claim 12, wherein closing the distal end of the container further comprises:
pulling on a tail of a purse string closure coupled with the distal end of the container.

20. The method of claim 12, further comprising:
using deployment structure to open and close the distal end of the container,
releasing the container and tissue contained therein from the deployment structure and the grasper, and
removing the tissue container and tissue contained therein from the patient as a unit separate from the deployment structure and the grasper.

21. The method of claim 12, further comprising:
using deployment structure to open and close the distal end of the container, and
using a plurality of one way tissue retainers carried on the deployment structure to retain tissue within the container after the grasper has pulled the tissue proximally past the tissue retainers and into the tissue container.
